(19) United States
(12) Patent Application Publication
(10) Pub. No.: US 2022/0047395 A1
(43) Pub. Date: Feb. 17, 2022

(54) KNEE JOINT ENDOPROSTHESIS APPARATUS AND KNEE JOINT ENDOPROSTHESIS

(71) Applicant: Aesculap AG, Tuttlingen (DE)

(72) Inventor: Arthur Bollinger, Engen (DE)

(21) Appl. No.: 17/470,324

(22) Filed: Sep. 9, 2021

Related U.S. Application Data
(63) Continuation of application No. PCT/EP2020/056768, filed on Mar. 13, 2020.

(30) Foreign Application Priority Data
Mar. 15, 2019 (DE) 10 2019 106 599.2

(51) Int. Cl. A61F 2/38; A61F 2/30

(52) U.S. Cl. CPC A61F 2/389; A61F 2/385; A61F 2/30734; A61F 2002/30133; A61F 2002/30332; A61F 2002/305; A61F 2002/30365

(57) ABSTRACT

A knee joint endoprosthesis apparatus includes a tibial component for anchoring to a tibia and a meniscal component. The tibial component and the meniscal component are coupled to one another in a coupling position and are completely separated from one another in a separating position. The tibial component has a top side defining an abutment plane, against which a bottom side of the meniscal component abuts in the coupling position. The knee joint endoprosthesis apparatus also includes a coupling device for coupling the tibial component and the meniscal component in the coupling position. The knee joint endoprosthesis apparatus is transferrable from the separating position into the coupling position by a relative movement of the tibial component and the meniscal component.

### CROSS-REFERENCE TO RELATED APPLICATIONS

This application is a continuation of international application number PCT/EP2020/056768 filed on Mar. 13, 2020 and claims the benefit of priority of German application number 10 2019 106 599.2 filed on Mar. 15, 2019. The contents of international application number PCT/EP2020/056768 and German application number 10 2019 106 599.2 are incorporated herein by reference in their entireties and for all purposes.

### FIELD

The present invention relates to knee joint endoprosthesis apparatuses comprising a tibial component for anchoring to a tibia, and comprising a meniscal component generally, and more specifically a knee joint endoprosthesis apparatus comprising a tibial component for anchoring to a tibia, and comprising a meniscal component, wherein the tibial component and the meniscal component are coupled to one another in a coupling position and are completely separated from one another in a separating position, which tibial component has a top side defining an abutment plane, against which top side a bottom side of the meniscal component abuts in the coupling position, wherein the knee joint endoprosthesis apparatus comprises a coupling device for coupling the tibial component and the meniscal component in the coupling position, wherein the knee joint endoprosthesis apparatus is transferrable from the separating position into the coupling position by a relative movement of the tibial component and the meniscal component.

Further, the present invention relates to knee joint endoprostheses generally, and more specifically to a knee joint endoprosthesis comprising a knee joint endoprosthesis apparatus and a femoral component that cooperates therewith.

### BACKGROUND

Knee joint endoprostheses of the kind described at the outset are known in different variants. Typically, first the femoral component and the tibial component of the knee joint endoprosthesis are anchored to the tibia and the femur, respectively, of a patient and then the meniscal component is introduced therebetween. In particular when the implantation of the knee joint endoprosthesis takes place while preserving collateral ligaments that stabilize the knee joint, in known solutions, an overextension of said ligaments is always necessary in order to introduce the meniscal component between the tibial component and the femoral component. In particular, this is independent of whether the meniscal component is to be immovably fixed to the tibia or is to be rotatably mounted thereon. The overextension of the ligaments should be avoided, however, in order to not impair a stability of the knee of the patient after the implantation of the knee joint endoprosthesis.

### SUMMARY

In a first aspect of the invention, a knee joint endoprosthesis apparatus comprising a tibial component for anchoring to a tibia, and comprising a meniscal component, wherein the tibial component and the meniscal component are coupled to one another in a coupling position and are completely separated from one another in a separating position, which tibial component has a top side defining an abutment plane, against which top side a bottom side of the meniscal component abuts in the coupling position, wherein the knee joint endoprosthesis apparatus comprises a coupling device for coupling the tibial component and the meniscal component in the coupling position, wherein the knee joint endoprosthesis apparatus is transferrable from the separating position into the coupling position by a relative movement of the tibial component and the meniscal component, wherein the coupling device is configured in such a way that the tibial component and the meniscal component, upon transferring the knee joint endoprosthesis apparatus from the separating position into the coupling position, are displaceable relative to one another in a direction parallel to the abutment plane from the separating position into an intermediate position and from the intermediate position in a direction transverse, in particular perpendicular, to the abutment plane and toward one another into the coupling position.

In a second aspect of the invention, a knee joint endoprosthesis comprising a knee joint endoprosthesis apparatus and a femoral component that cooperates therewith, wherein the knee joint endoprosthesis apparatus is configured in the form of a knee joint endoprosthesis apparatus comprising a tibial component for anchoring to a tibia, and comprising a meniscal component, wherein the tibial component and the meniscal component are coupled to one another in a coupling position and are completely separated from one another in a separating position, which tibial component has a top side defining an abutment plane, against which top side a bottom side of the meniscal component abuts in the coupling position, wherein the knee joint endoprosthesis apparatus comprises a coupling device for coupling the tibial component and the meniscal component in the coupling position, wherein the knee joint endoprosthesis apparatus is transferrable from the separating position into the coupling position by a relative movement of the tibial component and the meniscal component, wherein the coupling device is configured in such a way that the tibial component and the meniscal component, upon transferring the knee joint endoprosthesis apparatus from the separating position into the coupling position, are displaceable relative to one another in a direction parallel to the abutment plane from the separating position into an intermediate position and from the intermediate position in a direction transverse, in particular perpendicular, to the abutment plane and toward one another into the coupling position.

BRIEF DESCRIPTION OF THE DRAWING FIGURES

The foregoing summary and the following description may be better understood in conjunction with the drawing figures, of which:

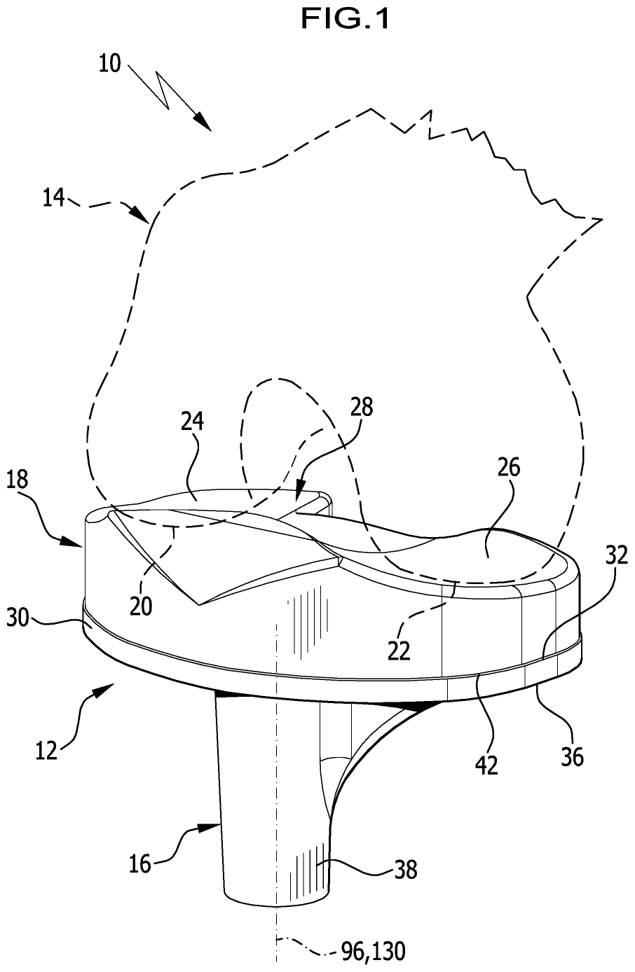

FIG. 1: shows a schematic perspective total view of an embodiment of a knee joint endoprosthesis;

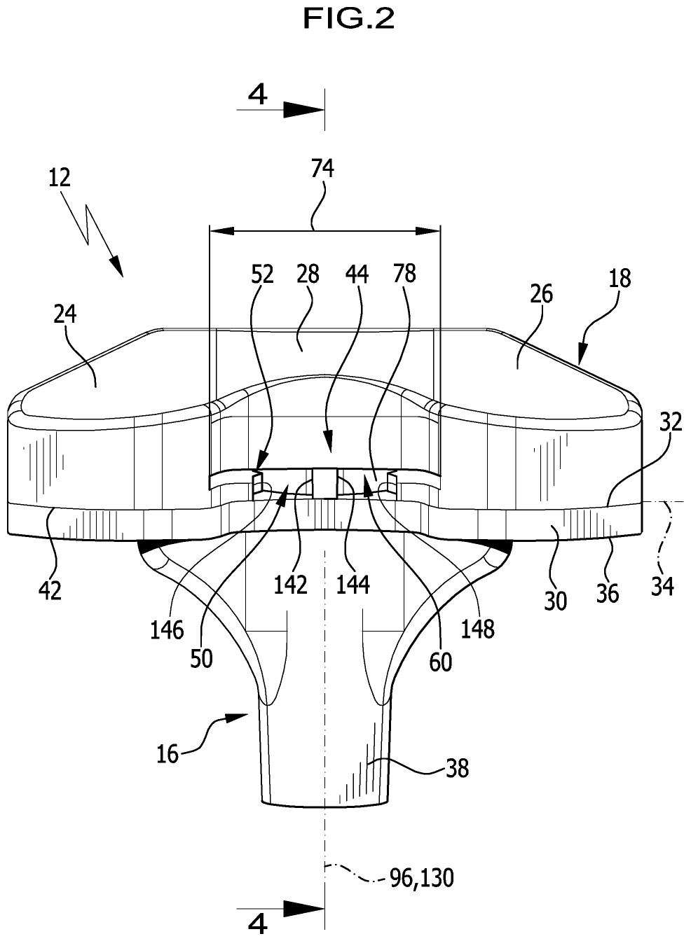

FIG. 2: shows a posterior view of a part of the embodiment, depicted in FIG. 1, of a knee joint endoprosthesis in the form of a knee joint endoprosthesis apparatus;

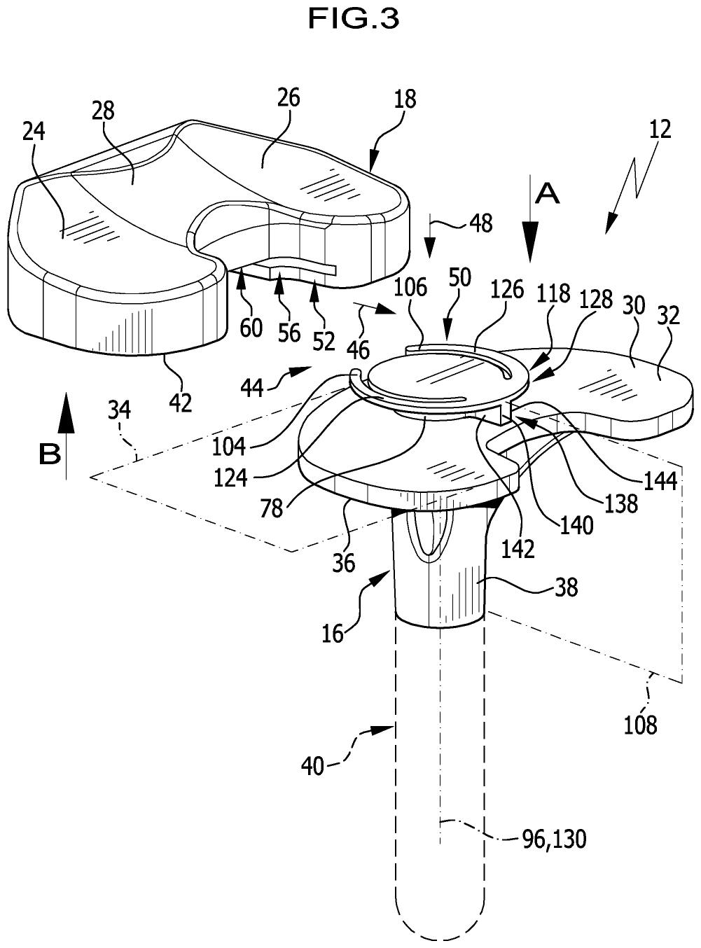

FIG. 3: shows an exploded depiction of the embodiment, depicted in FIG. 2, of a knee joint endoprosthesis apparatus in the separating position;

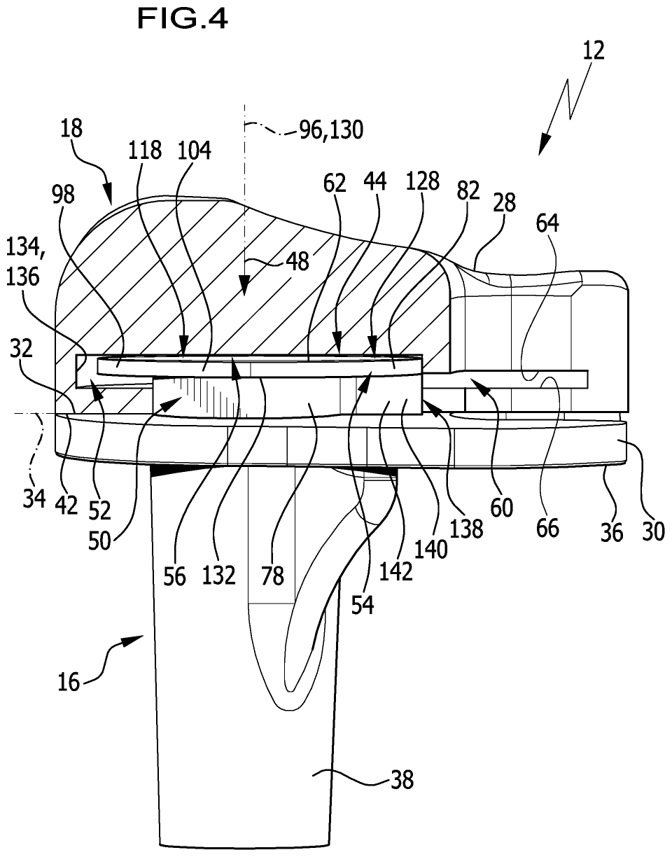

FIG. 4: shows a cut view along line 4-4 in FIG. 2;

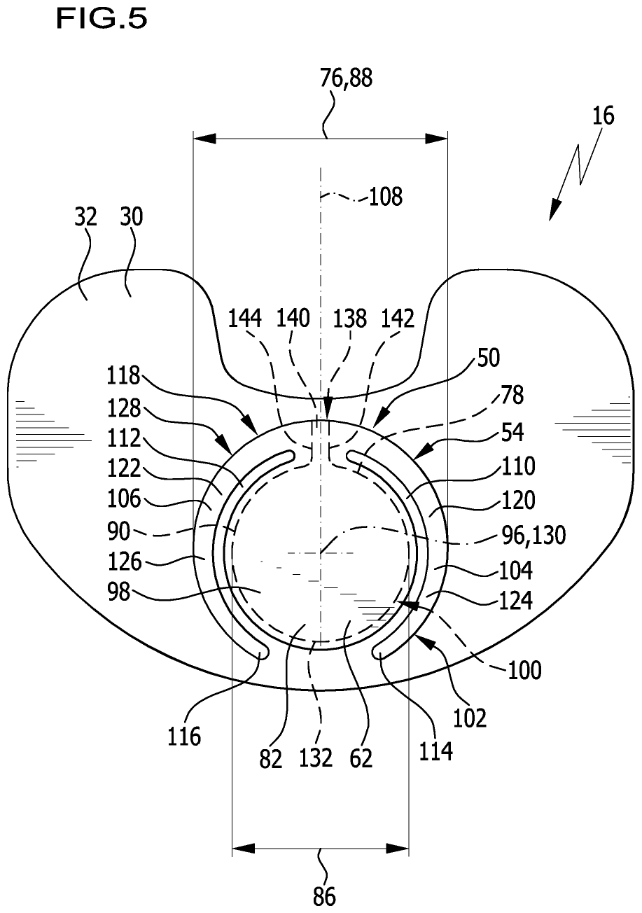

FIG. 5: shows a view of the tibial component of the embodiment of the knee joint endoprosthesis apparatus from FIG. 2 in the direction of the arrow A in FIG. 3;

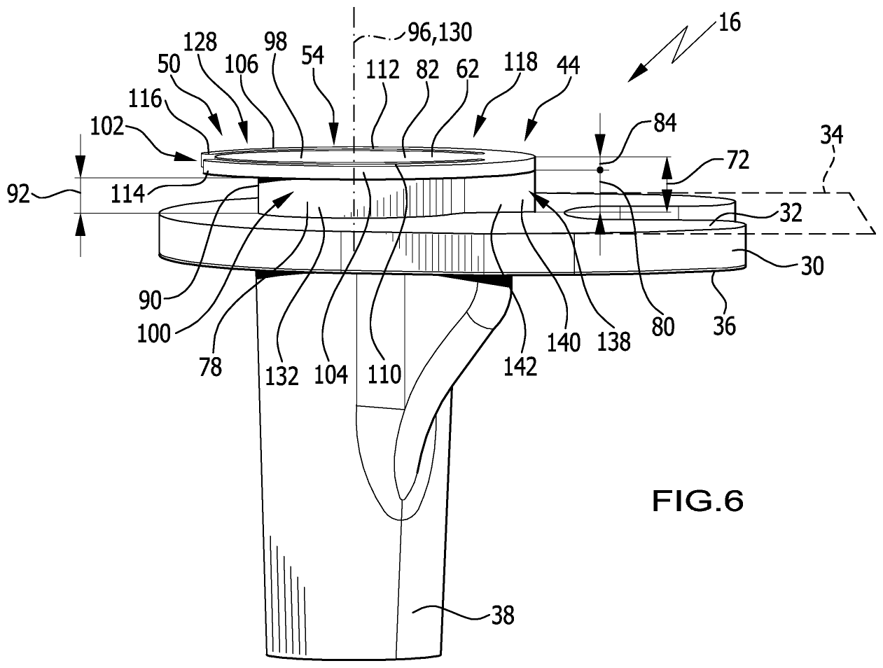

FIG. 6: shows a schematic perspective view of the tibial component from FIG. 5;

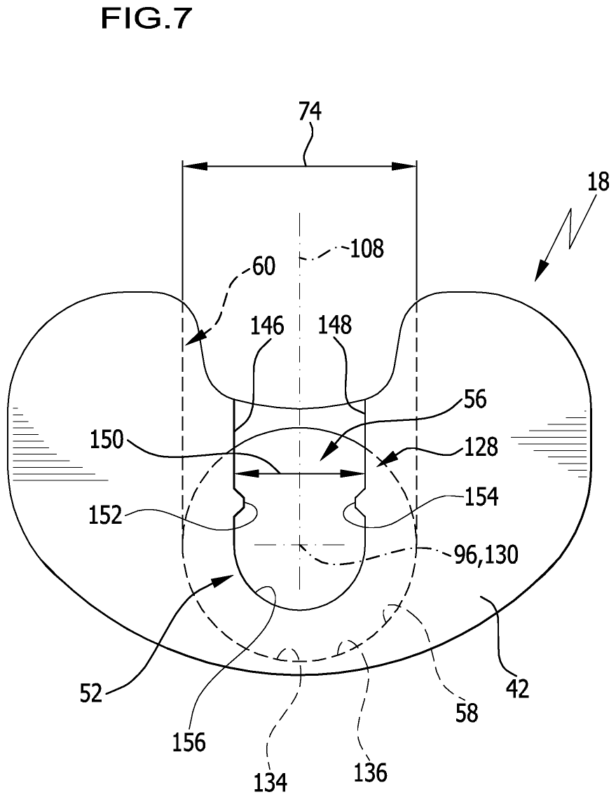

FIG. 7: shows a view of the meniscal component of the embodiment of the knee joint endoprosthesis apparatus from FIG. 2 in the direction of the arrow B in FIG. 3;

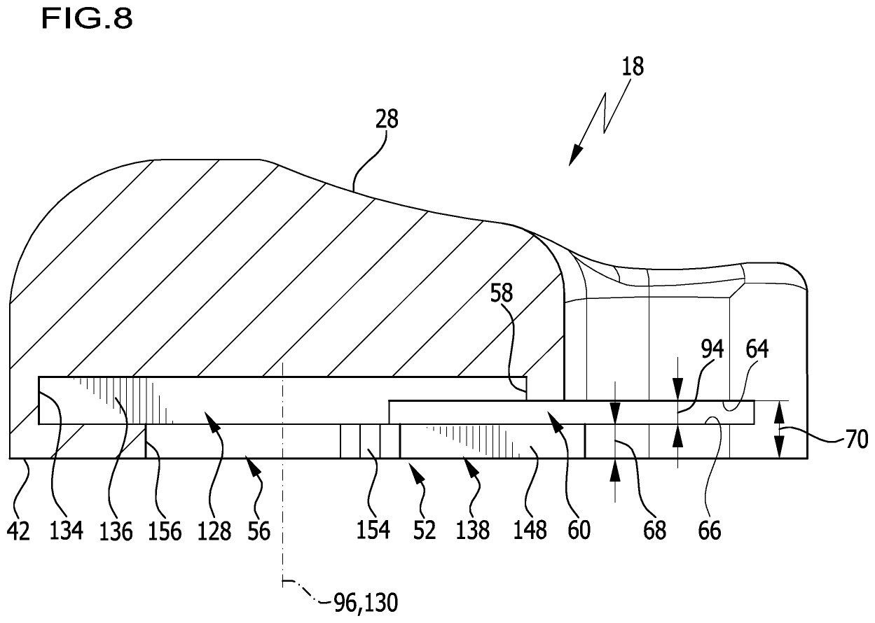

FIG. 8: shows a cut view of the meniscal component of the embodiment of the knee joint endoprosthesis apparatus from FIG. 2 along line 4-4;

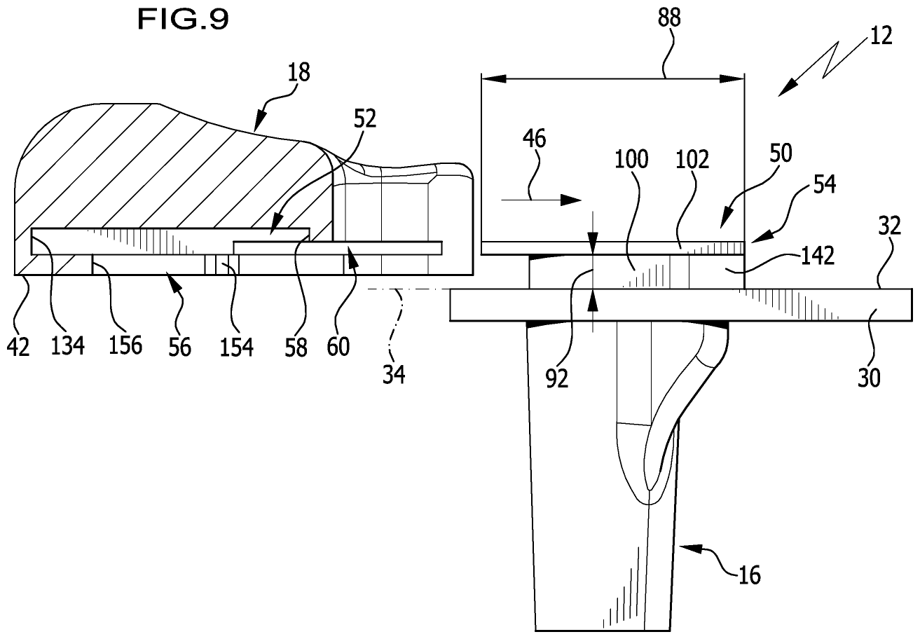

FIG. 9: shows a schematic, partially cut side view of a further embodiment of a knee joint endoprosthesis apparatus in the separating position;

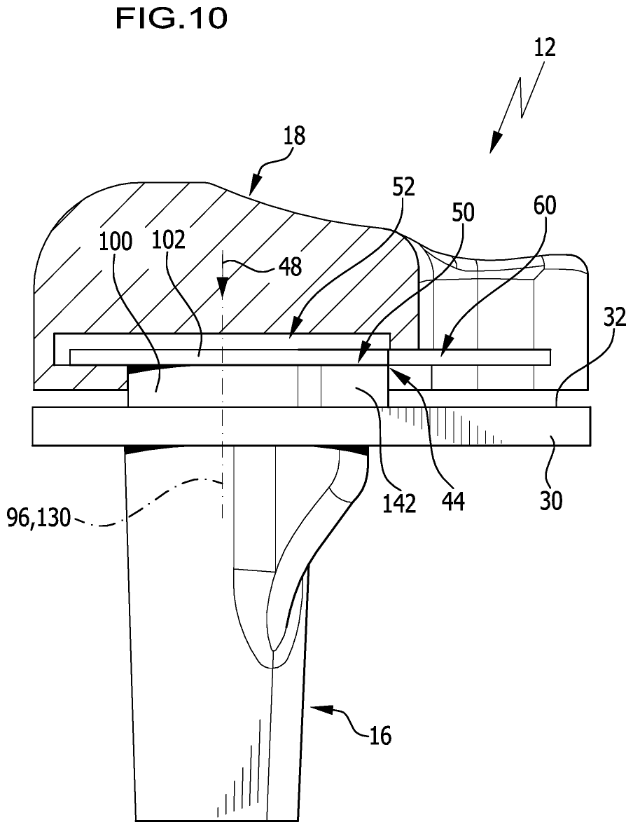

FIG. 10: shows a schematic, partially cut view of the knee joint endoprosthesis apparatus from FIG. 9 in the intermediate position;

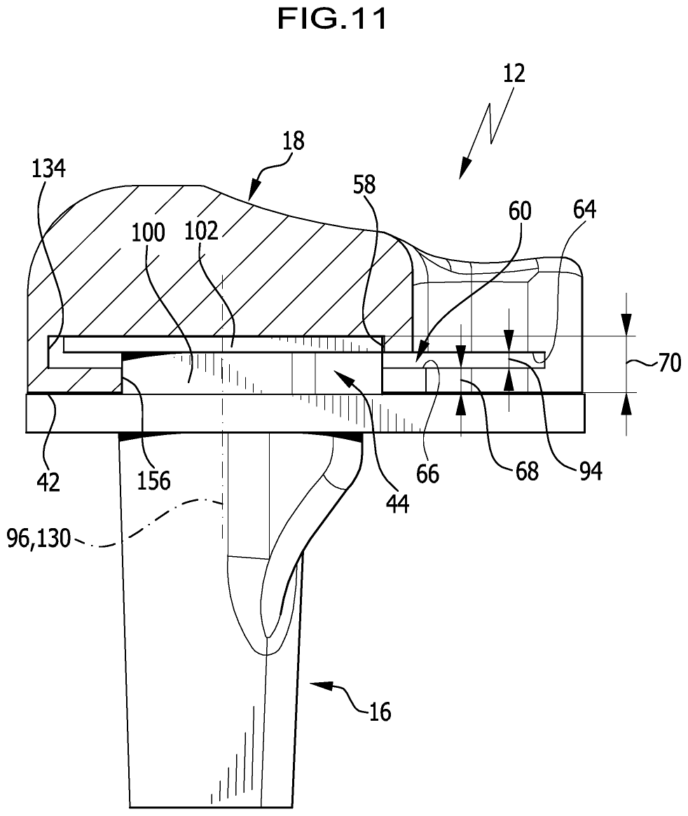

FIG. 11: shows a view of the knee joint endoprosthesis apparatus similar to FIG. 10 in the coupling position;

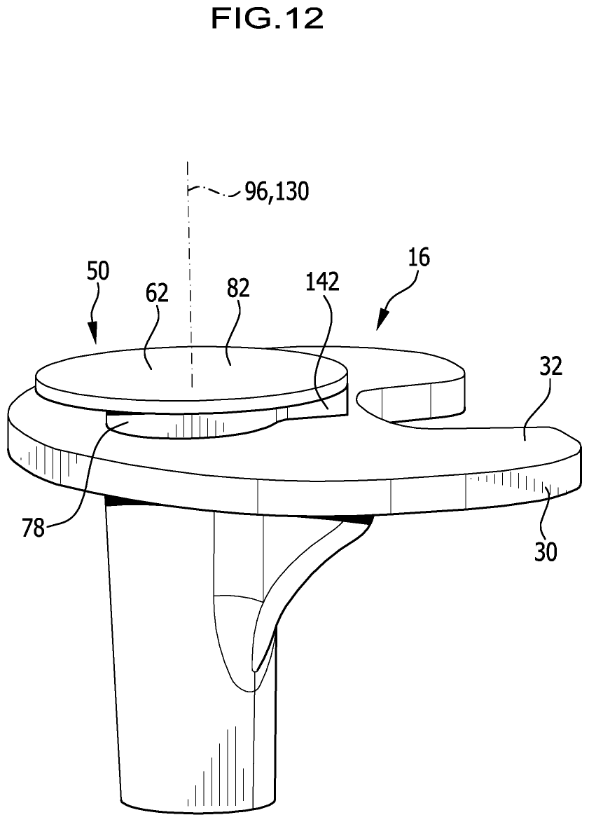

FIG. 12: shows a perspective view of the tibial component of the embodiment of the knee joint endoprosthesis apparatus from FIG. 9;

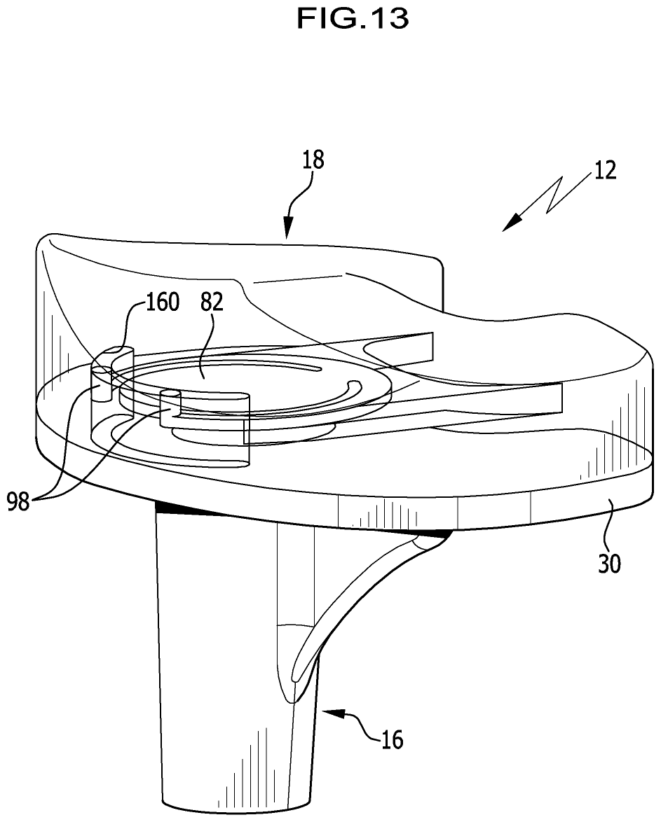

FIG. 13: shows a partially broken perspective total view of a further embodiment of a knee joint endoprosthesis apparatus in the coupling position;

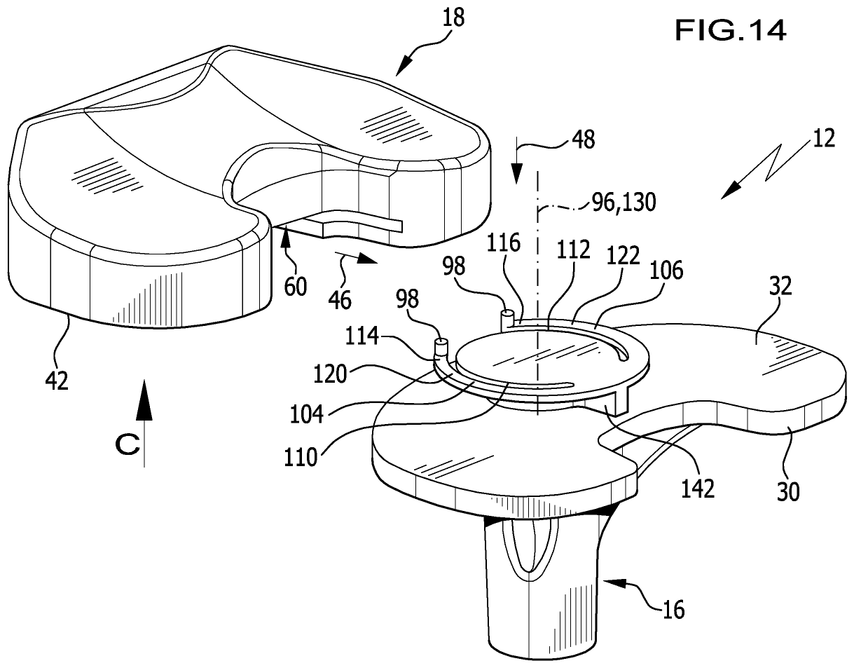

FIG. 14: shows a perspective view of the embodiment of a knee joint endoprosthesis apparatus from FIG. 13 in the separating position;

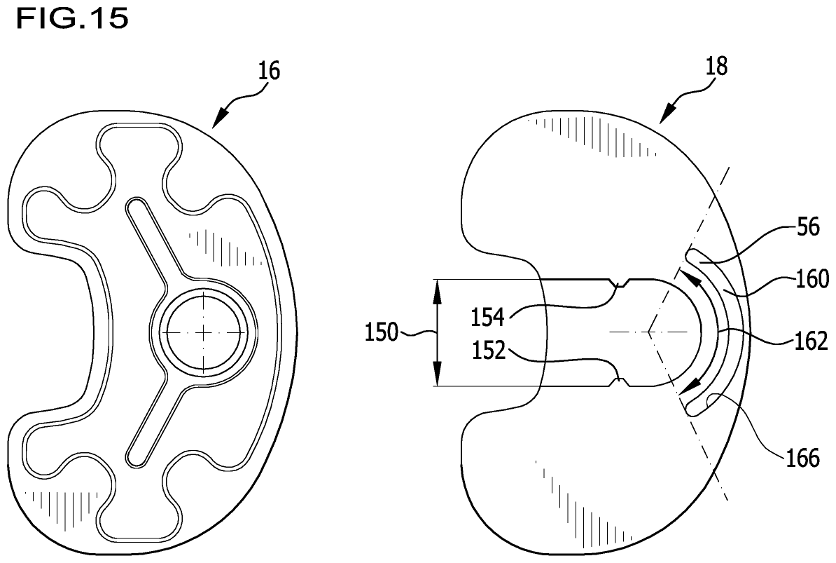

FIG. 15: shows a view of the embodiment of a knee joint endoprosthesis apparatus in the separating position in the direction of the arrow C in FIG. 14;

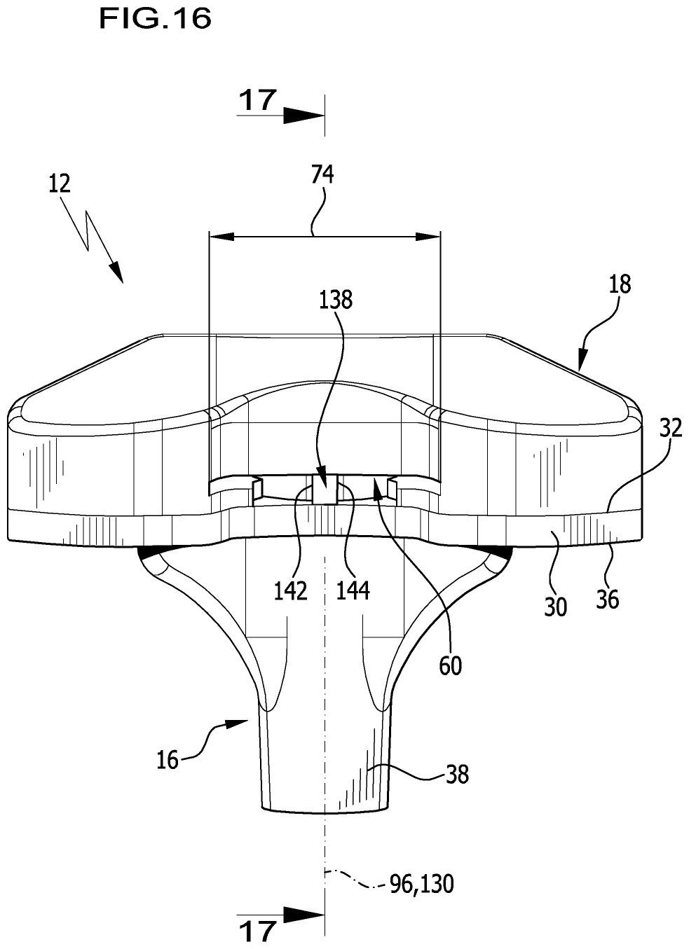

FIG. 16: shows a perspective anterior total view of the embodiment of the knee joint endoprosthesis apparatus from FIG. 13 in the coupling position; and

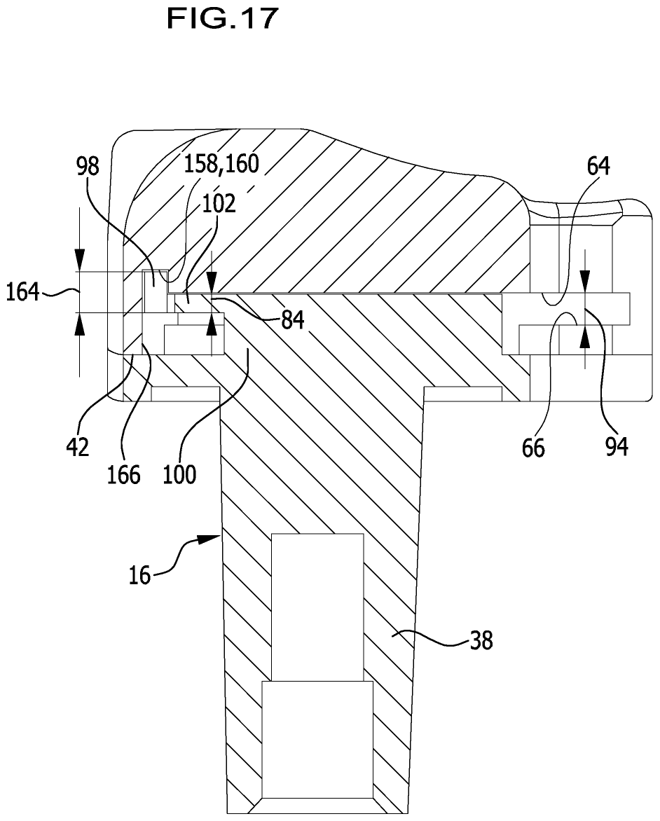

FIG. 17: shows a cut view along line 17-17 in FIG. 16.

### DETAILED DESCRIPTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a knee joint endoprosthesis apparatus comprising a tibial component for anchoring to a tibia, and comprising a meniscal component, wherein the tibial component and the meniscal component are coupled to one another in a coupling position and are completely separated from one another in a separating position, which tibial component has a top side defining an abutment plane, against which top side a bottom side of the meniscal component abuts in the coupling position, wherein the knee joint endoprosthesis apparatus comprises a coupling device for coupling the tibial component and the meniscal component in the coupling position, wherein the knee joint endoprosthesis apparatus is transferrable from the separating position into the coupling position by a relative movement of the tibial component and the meniscal component, wherein the coupling device is configured in such a way that the tibial component and the meniscal component, upon transferring the knee joint endoprosthesis apparatus from the separating position into the coupling position, are displaceable relative to one another in a direction parallel to the abutment plane from the separating position into an intermediate position and from the intermediate position in a direction transverse, in particular perpendicular, to the abutment plane and toward one another into the coupling position.

The further development, proposed in accordance with the invention, of known knee joint endoprosthesis apparatuses makes it possible, in particular, to insert the meniscal component between the femoral component and the tibial component, after the implantation of same, from the front, i.e., coming from the anterior, in particular without expanding or only minimally expanding the collateral ligaments maintained in the implantation. As a result of the possibility of displacing the meniscal component, in particular, in parallel to the abutment plane relative to the tibial component, said tibial component can be inserted from the front and thus in a manner that is gentle both on the ligaments and on the patient. In particular, this configuration of the knee joint endoprosthesis apparatus can be used both for knee joint endoprosthesis in which the meniscal component in the coupling position is held immovably relative to the tibial component or is rotatably and/or displaceably arranged relative thereto. Further, in the case of a knee joint endoprosthesis apparatus that has been developed further in the proposed way, the meniscal component can be exchanged again in a simple manner as required, for example when said meniscal component is damaged or a meniscal component selected by the surgeon is too high or too low.

It is favorable if the coupling device comprises at least one first coupling element and at least one second coupling element, if the at least one first coupling element is arranged or formed on the tibial component or on the meniscal component, if the at least one second coupling element is arranged or formed on the meniscal component or on the tibial component, if the at least one first coupling element and the at least one second coupling element are out of engagement in the separating position, and if the at least one first coupling element and the at least one second coupling element are in engagement in the coupling position. A coupling device of that kind can be formed in a simple manner and facilitates, in particular, the coupling of the meniscal component and the tibial component to one another.

It is advantageous if the at least one first coupling element is configured in the form of a coupling projection, if the at least one second coupling element is configured in the form of a coupling receptacle with a coupling recess, if the coupling receptacle is configured to accommodate the coupling projection in the intermediate position, and if the coupling recess is configured to accommodate at least a part of the coupling projection in the coupling position. The proposed design of the at least one first and second coupling elements makes it possible to engage and move the meniscal component and the tibial component relative to one another from the separating position into the intermediate position in a simple manner. Further, the coupling recess makes to possible to accommodate a part of the coupling projection in the coupling position. In particular, this is possible by a movement of the at least one part of the coupling projection from the intermediate position into the coupling recess, such that the knee joint endoprosthesis apparatus adopts the coupling position. This can be achieved, in particular, by the coupling recess being configured in the form of a depression or undercut that is formed on the coupling receptacle and faces away from the abutment plane. In addition, by means of the coupling recess, the meniscal component and the tibial component can be moved slightly toward one another upon the transition from the intermediate position into the coupling position.

The meniscal component can be secured to the tibial component in the coupling position in a simple manner if the coupling recess is open in a direction perpendicular to the abutment plane. In particular, the meniscal component and the tibial component can thus be moved toward one another perpendicularly to the abutment plane upon the transition from the intermediate position into the coupling position.

The coupling recess is preferably open exclusively in a direction perpendicular to the abutment plane. It can thus be ensured, in particular, that the movements of the meniscal component and the tibial component relative to one another from the separating position into the intermediate position on the one hand, and from the intermediate position into the coupling position on the other hand are linearly independent of one another in the mathematical sense. This makes it possible to secure the knee joint endoprosthesis apparatus in the coupling position in such a way that the meniscal component and the tibial component relative to one another are not releasable from one another only by a relative movement in parallel to the abutment plane. Further, a movement transverse to the abutment plane is still necessary in order to transfer the knee joint endoprosthesis apparatus first from the coupling position into the intermediate position.

It is advantageous if the meniscal component comprises the coupling recess, which is open facing in the direction toward the tibial component for inserting the at least one part of the coupling projection as the result of a relative movement from the intermediate position into the coupling position in the direction transverse, in particular perpendicular, to the abutment plane. Forming the coupling recess on the meniscal component has the advantage, in particular, that in the case in which the meniscal component is made of a plastic, no fragile projections have to be formed thereon in order to couple the meniscal component to a tibial component that is preferably made of a metallic material. The stability of the knee joint endoprosthesis apparatus overall can thus be improved.

A coupling of the meniscal component and the tibial component can be further simplified, in particular, by the meniscal component having an insertion opening for inserting the coupling projection into the coupling receptacle of the meniscal component. In particular, the insertion opening may be provided on the meniscal component in a rear or posterior region of the meniscal component, such that said meniscal component can be pushed onto the tibial component from the anterior, i.e., from the front.

It is favorable if the coupling projection has a coupling projection top side facing away from the abutment plane, if the insertion opening defines an upper delimiting face and a lower delimiting face, which extend in parallel to the bottom side of the meniscal component, if a distance of the lower delimiting face from the bottom side of the meniscal component is smaller than a distance of the upper delimiting face from the bottom side of the meniscal component, and if the distance of the lower delimiting face from the bottom side of the meniscal component is smaller than a distance of the coupling projection top side from the abutment plane. This configuration makes it possible, in particular, to displace the meniscal component relative to the tibial component in parallel to the abutment plane in order to transfer the knee joint endoprosthesis apparatus from the separating position into the intermediate position, wherein the bottom side of the meniscal component is thereby spaced somewhat at a distance from the top side. This distance between the bottom side of the meniscal component and the top side of the tibial component that the coupling device provides when the tibial component and the meniscal component are transferred from the separating position into the intermediate position then enables, in particular, a movement from the intermediate position into the coupling position by exactly this distance in such a way that the coupling projection can be inserted into the coupling recess.

It is favorable if the insertion opening defines an insertion width, if the coupling projection defines a coupling projection width, and if the coupling projection width corresponds to at least the insertion width. If the coupling projection is so wide that the coupling projection width corresponds to the insertion width, the meniscal component and the tibial component can be transferred from the separating position into the intermediate position substantially without noticeable resistance. If the coupling projection width is larger than the insertion width, though, either the insertion opening has to be slightly widened or the coupling projection has to be reduced slightly in its width upon transferring the knee joint endoprosthesis apparatus from the separating position into the intermediate position and vice versa. In particular, the meniscal component can in this way already be secured to the tibial component against unintentional release, because a defined insertion resistance then has to be overcome in order to separate the meniscal component and the tibial component from one another again. In addition, when coupling the meniscal component and the tibial component, a surgeon in this case obtains a haptic feedback when the knee joint endoprosthesis apparatus adopts the intermediate position, i.e., that position from which the knee joint endoprosthesis apparatus is transferrable into the coupling position by relative movement of the meniscal component and the tibial component in a direction transverse to the abutment plane.

The coupling projection width is preferably greater than the insertion width. In this case, as already described, in particular, a securing of the meniscal component and the tibial component can be achieved when the knee joint endoprosthesis apparatus adopts the intermediate position and could only be transferred from the intermediate position into the separating position by a movement, i.e., a displacement in parallel to the abutment plane.

In accordance with a further preferred embodiment of the knee joint endoprosthesis apparatus, provision may be made that at least one first and/or second moveable coupling elements are arranged or formed on the coupling projection and/or on the insertion opening, and that the coupling elements upon the transition from the separating position into the intermediate position are moveable for temporarily reducing the coupling projection width and/or for enlarging the insertion width. In particular, by means of this particular configuration of the coupling projection and/or the insertion opening, a latching or snapping connection can be formed, which makes it possible to snap in or latch the tibial component and the meniscal component relative to one another upon reaching the intermediate position commencing from the separating position.

The knee joint endoprosthesis apparatus can be formed in a simple and compact manner if the at least one first and/or second coupling elements are arranged or formed so as to be moveable in parallel to the abutment plane. In particular, they can thereby influence only a movement of the meniscal component and the tibial component relative to one another in parallel to the abutment plane, but not transverse, in particular perpendicular, to the abutment plane.

It is favorable if the at least one first and/or second coupling elements are deflectable against the action of a restoring device from a coupling element base position, in which the restoring device does not exert a restoring force on the at least one first and/or second coupling elements, into a coupling element deflected position. In this way, a snapping and/or latching connection device of the knee joint endoprosthesis apparatus for latching or snapping in the cooperating coupling elements upon reaching the intermediate position of the knee joint endoprosthesis apparatus can be achieved in a simple manner.

It is advantageous if the restoring device comprises at least one restoring element and if the at least one restoring element is associated with the at least one first and/or second coupling element. In particular, a restoring element may be associated with each coupling element. In this way, in particular, a coupling between the tibial component and the meniscal component can be achieved in a simple manner and in a way so as to be haptically perceptible to a surgeon.

The at least one first and/or second coupling element favorably comprises the at least one restoring element. This configuration enables, in particular, a compact structure of the knee joint endoprosthesis apparatus.

In order to enable a defined coupling between the tibial component and the meniscal component, it is favorable if a respective restoring element is associated with each of the at least one first and/or second coupling elements.

The at least one restoring element is favorably configured in the form of a spring element. Thus, in particular, a force that has to be applied in order to transfer the meniscal component and the tibial component relative to one another from the separating position into the intermediate position can be specified in a simple and defined manner.

It is advantageous if the coupling projection comprises at least one coupling member, if the coupling receptacle comprises at least one coupling member receptacle, and if in the coupling position the at least one coupling member engages into the at least one coupling member receptacle. In particular, the knee joint endoprosthesis apparatus may be configured in such a way that the at least one coupling member and the at least one coupling member receptacle are out of engagement in the intermediate position. The at least one coupling member and the at least one coupling member receptacle may, in particular, serve to secure the meniscal component and the tibial component in the coupling position. Further, in the case of a meniscal component that is moveably arranged relative to the tibial component, a movement can be restricted in a defined manner by appropriate design.

The knee joint endoprosthesis apparatus can be formed in a simple manner if the at least one coupling member is arranged or formed projecting from the at least one coupling element. In particular, it may be arranged or formed projecting from the at least one coupling element transversely to the abutment plane.

It is favorable if the at least one coupling member projects facing away from the at least one coupling element in a direction transverse, in particular perpendicular, to the abutment plane. In particular, it may be arranged or formed projecting facing away from the abutment plane. The coupling member receptacle may, in particular, also form the coupling recess and the at least one coupling member may accommodate the at least one part of the coupling projection, which are in engagement in the coupling position.

It is advantageous if the coupling member receptacle is configured in the form of a coupling member receptacle groove that is open facing in the direction toward the abutment plane. Such a design makes it possible, in particular, to configure the coupling member receptacle in the form of a guidance element in which the at least one coupling member is guided in a defined manner upon a relative movement between the tibial component and the meniscal component. For example, the coupling member receptacle groove may be of rectilinear or curved configuration.

It is favorable if the coupling projection comprises at least one first portion, which extends directly away from the top side of the tibial component, and a second portion adjoining the first portion, and if a maximum extent of the first portion in a plane parallel to the abutment plane is smaller than a maximum extent of the second portion in a plane parallel to the abutment plane. By means of this design, in particular, an undercut can be formed on the coupling projection in a simple manner, namely in the region of the first portion. The coupling receptacle may be correspondingly configured and have regions that, in the intermediate position and/or in the coupling position, engage between the second portion and the top side of the tibial component, i.e., in the region of the first portion.

In order to be able to form a knee joint endoprosthesis that is able to replicate a movement of a knee joint that is as natural as possible, it is favorable if the meniscal component and the tibial component are moveable relative to one another in the coupling position. In particular, they may be rotatably and/or displaceably held on one another.

In accordance with a further preferred embodiment, provision may be made that the coupling device forms a rotary bearing device for rotatably mounting the meniscal component and the tibial component relative to one another in the coupling position. This design enables, in particular, a compact configuration of the knee joint endoprosthesis apparatus, because the coupling device simultaneously takes on the function of the rotary bearing device.

A structure of the knee joint endoprosthesis apparatus can be realized in a particularly compact manner, in particular, by the rotary bearing device comprising the at least one first coupling element and the at least one second coupling element.

It is advantageous if the at least one first coupling element and the at least one second coupling element are formed rotationally symmetrical or substantially rotationally symmetrical and define an axis of rotation, in relation to which the meniscal component and the tibial component in the coupling position are rotatably mounted relative to one another. An axis of rotation can be achieved in a simple manner through a rotationally symmetrical or substantially rotationally symmetrical configuration of the at least one first or the at least one second coupling element.

The axis of rotation preferably extends transversely, in particular perpendicularly, to the abutment plane. In this way, it can be achieved, in particular, that the bottom side of the meniscal component, upon a rotation relative to the tibial component, can abut in surface-to-surface contact against the top side of the tibial component, namely independently of which rotation position the meniscal component and the tibial component adopt relative to one another.

It is advantageous if the at least one first and/or second coupling elements are arranged or formed so as to be displaceable and/or pivotable away from the axis of rotation or towards the axis of rotation. Coupling elements of that kind make it possible in a simple manner to temporarily reduce a coupling projection width upon transferring the knee joint endoprosthesis apparatus from the separating position into the intermediate position and vice versa.

The coupling member receptacle groove is advantageously arranged or formed concentrically surrounding the axis of rotation. This makes it possible, in particular, to configure the coupling member receptacle groove as an element for limiting a rotation of the meniscal component and the tibial component relative to one another, i.e., in particular as part of a rotation limiting device. Further, as already described, a relative movement of the tibial component and the meniscal component can also be guided in a defined manner by the coupling member receptacle groove. The coupling member receptacle groove may thus form, e.g., a part of the rotary bearing device.

It is favorable if an undercut that is open in parallel to the abutment plane is formed between the top side of the tibial component and the second portion in the region of the first portion. In particular, said undercut may be configured in the form of a groove that faces away from the axis of rotation and is all-round at least in sections, in particular completely. In particular, a movement of the meniscal component and the tibial component away from one another and toward one another can be limited in a defined manner by the undercut.

It is favorable if the coupling receptacle has at least one undercut that is open in parallel to the bottom side of the meniscal component and facing in the direction toward the axis of rotation. In particular, said undercut may be configured in the form of a groove that is all-round at least in sections, in particular completely, and faces toward the axis of rotation. This design makes it possible, in particular, to insert the second portion of the coupling projection into this undercut during the transition from the separating position into the intermediate position. In particular, the undercut may be configured in such a way that coupling elements on the coupling projection engage into the undercut after the coupling projection has passed through the insertion opening.

In accordance with a further preferred embodiment, provision may be made that the knee joint endoprosthesis apparatus comprises a rotation limiting device for limiting a rotation of the meniscal component and the tibial component relative to one another in the coupling position. Thus, in particular, a relative movement between the meniscal component and the tibial component can be restricted in a defined manner by the rotation limiting device. In the case of a knee joint, such a rotation defines, in particular, a rotation about a leg longitudinal axis of the patient.

It is favorable if the rotation limiting device comprises at least one first stop face arranged or formed on the tibial component and at least one second stop face that cooperates therewith and is arranged or formed on the meniscal component, and if the at least one first stop face and the at least one second stop face abut against one another in a rotation position deflected from a base position by a maximum rotation angle. With such a rotation limiting device, a rotation of the meniscal component and the tibial component relative to one another in relation to the axis of rotation can be limited in a simple manner. In particular, the rotation limiting device can limit a rotational movement in mutually opposite directions of rotation in a defined manner by means of corresponding stop faces on the meniscal component and the tibial component. For example, a rotation angle from an undeflected position may be about 30 degrees both in the one direction as well as in the other direction.

The knee joint endoprosthesis apparatus can be configured in a simple and compact manner if the at least one first stop face and the at least one second stop face extend transversely, in particular perpendicularly, to the abutment plane. In particular, stopping forces can thus be absorbed without this resulting in a relative movement of the tibial component and the meniscal component in a direction perpendicular to the abutment plane.

It is advantageous if the meniscal component has a meniscal component top side, which has medial and lateral meniscal joint faces. These meniscal joint faces can, in particular, cooperate with corresponding femoral joint faces on a femoral component of a knee joint endoprosthesis.

The present invention further relates to a knee joint endoprosthesis comprising a knee joint endoprosthesis apparatus and a femoral component that cooperates therewith, wherein the knee joint endoprosthesis apparatus is configured in the form of a knee joint endoprosthesis apparatus comprising a tibial component for anchoring to a tibia, and comprising a meniscal component, wherein the tibial component and the meniscal component are coupled to one another in a coupling position and are completely separated from one another in a separating position, which tibial component has a top side defining an abutment plane, against which top side a bottom side of the meniscal component abuts in the coupling position, wherein the knee joint endoprosthesis apparatus comprises a coupling device for coupling the tibial component and the meniscal component in the coupling position, wherein the knee joint endoprosthesis apparatus is transferrable from the separating position into the coupling position by a relative movement of the tibial component and the meniscal component, wherein the coupling device is configured in such a way that the tibial component and the meniscal component, upon transferring the knee joint endoprosthesis apparatus from the separating position into the coupling position, are displaceable relative to one another in a direction parallel to the abutment plane from the separating position into an intermediate position and from the intermediate position in a direction transverse, in particular perpendicular, to the abutment plane and toward one another into the coupling position.

The knee joint endoprosthesis then has the advantages that were already described above in conjunction with preferred embodiments of knee joint endoprosthesis apparatuses.
It is advantageous if the femoral component comprises medial and lateral femoral joint faces facing in the direction toward the meniscal component, which femoral joint faces in an implantation position of the knee joint endoprosthesis abut against medial and lateral meniscal joint faces of the meniscal component and cooperate therewith upon a relative movement of the femoral component and the meniscal component. In this way, in particular, a movement of the femoral component and the meniscal component relative to one another can be achieved that is very similar to a relative movement of a natural tibia relative to the natural femur.

A first embodiment of a knee joint endoprosthesis 10 is depicted in FIG. 1. It comprises a first embodiment of a knee joint endoprosthesis apparatus 12 and a first embodiment of a femoral component 14 that is schematically drawn dashed in FIG. 1.

The knee joint endoprosthesis apparatus 12 comprises a tibial component 16 for anchoring to a tibia of a patient, and comprises a meniscal component 18.

The femoral component 14 is configured to be fixed to a femur of a patient. It comprises medial and lateral femoral joint faces 20 and 22, which face in the direction toward the meniscal component 18 and in an implantation position abut against corresponding medial and lateral meniscal joint faces 24 and 26, respectively, of the meniscal component 18 and cooperate therewith upon a relative movement of the femoral component 14 and meniscal component 18.

The medial and lateral meniscal joint faces 24 and 26 are arranged or formed on a meniscal component top side 28 that faces in the direction toward the femoral component 14.

The tibial component 16 comprises a tibial plate 30, which has a top side 32 that is planar and defines an abutment plane 34. Arranged projecting from a bottom side 36 of the tibial plate 30 is a shank 38, which may optionally be extended to a required length with shank extensions 40 that are suitable for the patient for inserting into a medullary canal of the tibia of the patient, so that the tibial component 16 is securely fixable to the tibia of the patient.

The tibial plate 30 is substantially kidney-shaped in a plan view, as is depicted for example in FIG. 5.

The tibial component 16 is made of a metallic material, for example of an implant steel or titanium.

The meniscal component has a planar bottom side 42 which, in a coupling position of the knee joint endoprosthesis apparatus 12 in which the tibial component 16 and the meniscal component 18 are coupled to one another, abuts against the top side 32. The bottom side 42 has in a plan view, as is depicted schematically in FIG. 7, an identical shape to the top side 32 so that the meniscal component 18 in an undeflected base position completely covers the tibial plate 30.

For coupling the tibial component 16 and the meniscal component 18, the knee joint endoprosthesis apparatus 12 comprises a coupling device 44.

The knee joint endoprosthesis apparatus 12 is configured in such a way that it is transferrable from a separating position, in which the tibial component 16 and the meniscal component 18 are completely separated from one another, as schematically depicted in FIG. 3, into the coupling position by a relative movement of the tibial component 16 and the meniscal component 18. In particular, the coupling device 44 is configured in such a way that the tibial component 16 and the meniscal component 18, upon transferring the knee joint endoprosthesis apparatus 12 from the separating position into the coupling position, can first be transferred relative to one another in a direction parallel to the abutment plane 34 from the separating position into an intermediate position. This movement is symbolized in FIG. 3 by the arrow 46, which is oriented in parallel to the abutment plane 34.

The knee joint endoprosthesis apparatus 12 can be transferred from the intermediate position into the coupling position, namely by a movement in a direction transverse, symbolized by the arrow 48, to the abutment plane 34, so that the meniscal component 18 and the tibial component 16 are moved toward one another until the bottom side 42 abuts against the top side 32.

To enable this function of the coupling device 44, same comprises a first coupling element 50 on the tibial component 16 and a corresponding second coupling element 52 on the meniscal component 18. The first and second coupling elements 50 and 52 are out of engagement in the separating position and are in engagement in the coupling position.

In the embodiment of the knee joint endoprosthesis apparatus 12 depicted in FIGS. 1 to 8, the first coupling element 50 is configured in the form of a coupling projection 54. The second coupling element 52 is configured in the form of a coupling receptacle 56.

The coupling receptacle 56 on the meniscal component 18 further comprises a coupling recess 58 in the form of a recess.

The coupling receptacle 56 is configured to accommodate the coupling projection 54 in the intermediate position. In other words, the tibial component 16 and the meniscal component 18 can be transferred starting from the separating position into the intermediate position, wherein the coupling projection 54 is then inserted into the coupling receptacle 56.

The coupling recess 58 in the coupling position accommodates a part of the coupling projection 54. To make this possible, namely upon a movement of the meniscal component 18 and the tibial component 16 toward one another, the coupling recess 58 is open in a direction perpendicular to the abutment plane 34. In other words, the coupling recess 58 forms a depression or undercut of the coupling receptacle 56 that is set back from the abutment plane 34.

The coupling projection 54 comprises a cylindrical coupling body 78, which has a height 80 in relation to the top side 32 and defines a first portion 100. The coupling projection 54 further comprises a coupling disc 82, which is arranged on the coupling body 78, has a thickness 84, and defines a second portion 102. A diameter 86 of the coupling body 78 and thus a maximum extent thereof in a plane parallel to the abutment plane 34 is smaller than a diameter 88 of the second portion 102. Thus, formed between the coupling disc 82 and the tibial plate is a groove 90 that is all-round in relation to a longitudinal axis 96 of the coupling body 78, which extends perpendicularly to the abutment plane 34, and is formed open facing away from the longitudinal axis 96, the width 92 of said groove 90 corresponding to the height 80.

The coupling disc 82 of the coupling projection 54 has a coupling projection top side 62 that faces away from the abutment plane 34.

In order to be able to insert the coupling projection 54 into the coupling receptacle 56, the meniscal component 18 has an insertion opening 60. The coupling projection 54 can be inserted through said insertion opening 60 into the coupling receptacle 56.

The insertion opening 60 of the meniscal component 18 defines an upper delimiting face 64 and a lower delimiting face 66. Both delimiting faces 64 and 66 extend in parallel to the bottom side 42 of the meniscal component 18.

A distance of 68 the lower delimiting face 66 from the bottom side 42 is smaller than a distance 70 of the upper delimiting face 64 from the bottom side 42. Further, a distance 72 of the coupling projection top side 62 from the abutment plane 34 is greater than the distance 68. A width 94 of the insertion opening 60 in parallel to the longitudinal axis 96 corresponds to a difference of the distances 70 and 68 and is insignificantly greater than the thickness 84 of the second portion 102, such that the coupling disc 82 can be inserted in parallel to the abutment plane 34 through the insertion opening 60 that is slot-shaped seen from the posterior, as schematically depicted in FIG. 2.

The described relationship between the distance 68 and the distance 72 results in the bottom side 42, upon transferring the knee joint endoprosthesis apparatus 12 from the separating position into the intermediate position, being spaced at a distance, namely a distance that corresponds to a difference between the distances 72 and 68.

The insertion opening 60 defines an insertion width 74. The coupling projection 54 defines a coupling projection width 76 in the region of the coupling disc 82. In the embodiment of the knee joint endoprosthesis apparatus 12 depicted in FIGS. 1 to 8, the coupling projection width 76 is greater than the insertion width 74.

In order to be able to pass the coupling projection 54 through the insertion opening 60, the coupling projection width 76 must be temporarily reduced. This is achieved by the second portion 102 comprising two moveable coupling elements 104 and 106, which, like the coupling projection 54, are formed mirror symmetrical to a mirror plane 108 that contains the longitudinal axis 96 and extends in the anterior-posterior direction.

The coupling elements 104 and 106 are each formed by a respective semicircular slot 110 and 112, respectively, in the second portion 102, said slot extending concentrically to the longitudinal axis 96. Free ends 114 and 116 of the coupling elements 104 and 106 are slightly at a distance from one another and are positioned on the anterior side on the coupling projection 54.

The coupling elements 104 and 106 are arranged or formed so as to be moveable in parallel to the abutment plane 34.

The coupling elements 104 and 106 are deflectable against the action of a restoring device 118 from a coupling element base position, in which the restoring device 118 does not exert a restoring force on the coupling elements 104 and 106, into a coupling element deflected position. The coupling elements 104 and 106 are, in particular, moveable toward one another with their free ends 114 and 116 when said coupling elements 104 and 106 are passed through the slot-shaped insertion opening 60. The coupling projection width 76 is thereby temporarily slightly reduced.

As soon as the second portion 102 is inserted into the coupling receptacle 56, the coupling elements 104 and 106 can pivot out back into their original coupling element base position. In this way, the coupling projection 54 is snapped into the coupling receptacle 56.

The restoring device 118 comprises two restoring elements 120 and 122, which are each associated with a coupling element 104 and 106, respectively. In the embodiment of the knee joint endoprosthesis apparatus 12 depicted in FIGS. 1 to 8, each coupling element 104, 106 comprises a respective restoring element 120 and 122, respectively.

The restoring elements 120 and 122 are each configured in the form of a spring element 124 and 126, respectively.

The meniscal component 18 and the tibial component 16 are moveable relative to one another in the coupling position. They can be rotated relative to one another about the longitudinal axis 96. This is made possible by the coupling device 44, which comprises or forms a rotary bearing device 128 for rotatably mounting the meniscal component 18 and the tibial component 16 relative to one another in the coupling position.

The rotary bearing device 128 comprises the two coupling elements 50 and 52.

The first coupling element 50 and the second coupling element 52 are each formed rotationally symmetrical or substantially rotationally symmetrical and define an axis of rotation 130, which coincides with the longitudinal axis 96. The meniscal component 18 and the tibial component 16 in the coupling position are mounted on one another so as to be rotatable relative to one another with respect to the axis of rotation 130.

The axis of rotation 130 extends perpendicularly to the abutment plane 34.

As already described, the coupling elements 104 and 106 are formed or arranged so as to be pivotable toward the axis of rotation 130.

The groove 90 formed between the top side 32 of the tibial component 16 and the second portion 102 in the region of the first portion 100 forms an undercut 132 that is open in parallel to the abutment plane 34.

The coupling receptacle 56 on the meniscal component 18 has an undercut 134 that is open in parallel to the bottom side 42 thereof and facing in the direction toward the longitudinal axis 96 or the axis of rotation 130. Said undercut 134 is configured in the form of a groove 136 that faces toward the axis of rotation 130 in sections.

The described embodiment of the knee joint endoprosthesis apparatus 12 further comprises a rotation limiting device 139 for limiting a rotation of the meniscal component 18 and the tibial component 16 relative to one another in the coupling position.

The rotation limiting device 138 comprises a stop 140, which is configured in the form of a projection that projects from the first portion 100 pointing in the posterior direction and defines two stop faces 142 and 144 facing away from one another.

Interacting with the stop faces 142 and 144 are stop faces 146 and 148 of the meniscal component 18 facing toward one another, which delimit the insertion opening 60 between the lower delimiting face 66 and the bottom side 42. A distance 150 between the stop faces 146 and 148 is slightly larger than the diameter 86 of the first portion 100, so that said first portion 100 can be inserted through the insertion opening 60 into the coupling receptacle 56 upon coupling the meniscal component 18 and the tibial component 16 to one another.

The stop face 142 delimits with the stop face 146 a movement of the meniscal component 18 relative to the tibial component 16 in the counter-clockwise direction, seen in plan view of the top side 32. The cooperating stop faces 144 and 148 correspondingly delimit a rotational movement of the meniscal component 18 relative to the tibial component 16 about the axis of rotation 130 in the clockwise direction.

The two rotation positions maximally deflected as described, in which positions the stop faces 142 and 146, on the one hand, and the stop faces 144 and 148, on the other hand, abut against one another, delimit a maximum rotation angle.

The stop faces 142, 144 and 146, 148 extend perpendicularly to the abutment plane 34.

Formed on the meniscal component 18 are small lobes 152 and 154 that project from the stop faces 146 and 148, respectively, and slightly reduce the distance 150 between the stop faces 146 and 148

Upon inserting the coupling projection 54 through the insertion opening 60 into the coupling receptacle 56, the first portion 100 must be pushed between the lobes 152 and 154. This leads to a temporary deformation of the meniscal component 18, which is made of a plastic, in particular polyethylene with an ultrahigh molecular weight (UBMWPE), said deformation being perceptible as a clicking noise. In the coupling position, the first portion 100 is then secured between the lobes 152 and 154 as well as in a concave rim 156 that faces toward the axis of rotation 130.

A further embodiment of a knee joint endoprosthesis apparatus 12 is depicted for example in FIGS. 9 to 12. It substantially corresponds in its structure to the embodiment of the knee joint endoprosthesis apparatus 12 depicted in FIGS. 1 to 8 and described above. Therefore, for the sake of clarity, identical or similar components are provided with the same reference numerals in the embodiment of the knee joint endoprosthesis apparatus 12 depicted in FIGS. 9 to 12 as in the embodiment of the knee joint endoprosthesis apparatus 12 depicted in FIGS. 1 to 8.

Unlike in the embodiment of FIGS. 1 to 8, no coupling elements 104, 106 are provided in the embodiment of FIGS. 9 to 12. The second portion 102, as can be seen in FIG. 12, is configured in the form of a circular coupling disc 82.

So that the second portion 102 can be inserted through the insertion opening 60 between the upper delimiting face 64 and the lower delimiting face 66, the insertion width 74 on the meniscal component 18 is slightly larger than the diameter 88 of the coupling disc 82.

For coupling the tibial component 16 and the meniscal component 18 of the embodiment of the knee joint endoprosthesis apparatus 12 depicted in FIGS. 9 to 12, the meniscal component 18 is displaced in parallel to the abutment plane 34 with its bottom side 42 somewhat at a distance from the top side 32 and the second portion 102 is inserted between the delimiting faces 64 and 66 of the insertion opening 60. This means that the meniscal component 18 is pushed from the anterior in the posterior direction onto the tibial component 16. As soon as the first portion 100 has passed the two lobes 152 and 154, a clicking sound can be heard. As described above, the coupling projection 54 is then secured in the coupling receptacle 56 in the intermediate position.

From this intermediate position, the second portion 102, i.e., a part of the coupling projection 54, can dip into the coupling recess 48 by a movement of the meniscal component 18 in the direction toward the top side 32 of the tibial plate 30. The meniscal component 18 and the tibial component 16 can now, insofar as the rotation limiting device 138 allows, be rotated relative to one another about the axis of rotation 130.

The meniscal component 18 is secured to the tibial component 16 by the engagement of the coupling disc 82 into the coupling recess 58. A displacement of the meniscal component 18 relative to the tibial component 16 in parallel to the abutment plane 34 is, in this coupling position schematically depicted in FIG. 11, no longer possible or possible only to a very limited extent.

To remove the meniscal component 18 from the tibial component 16, in reverse order, first the meniscal component 18 must be raised in relation to the top side 32 in order to transfer the knee joint endoprosthesis apparatus 12 into the intermediate position. From this intermediate position that is schematically depicted in FIG. 10, the second portion 102 can then be guided back through the insertion opening 60 in the region between the delimiting faces 64 and 66 until the knee joint endoprosthesis apparatus 12 readopts the separating position schematically depicted in FIG. 9.

A further embodiment of a knee joint endoprosthesis apparatus 12 is schematically depicted in FIGS. 13 to 17. It corresponds in its structure to a large extent with the embodiment of the knee joint endoprosthesis apparatus 12 described in conjunction with FIGS. 1 to 8. Identical components of the knee joint endoprosthesis apparatus 12 in FIGS. 13 to 17 are therefore designated with the same reference numerals as in the two embodiments described above.

Unlike in the embodiment of the knee joint endoprosthesis apparatus 12 of FIGS. 1 to 8, the coupling projection 54 comprises two coupling members 98. Said coupling members 98 project in the form of short cylindrical projections from the free ends 114 and 116 of the coupling elements 104 and 106 oriented in parallel to the longitudinal axis 96 and facing away from the top side 32 of the tibial plate 30.

Formed on the meniscal component 18 is a groove 158 that is formed concentrically to the longitudinal axis 96 and open facing in the direction toward the top side 32, said groove 158 forming a coupling member receptacle 160. The two coupling members 98 in the coupling position engage into this groove 158, which extends symmetrically to the mirror plane 108 over an angular range 162 of about 120°. The coupling member receptacle 160 thus also forms the coupling recess 58.

A total height 164 of the coupling members 98 in parallel to the longitudinal axis 96, which also comprises the thickness 84, requires that the width 94 of the insertion opening 60 between the delimiting faces 64 and 66 is slightly larger than the total height 164. The width 94 in the embodiment of the knee joint endoprosthesis apparatus 12 of FIGS. 13 to 17 is thus greater than the width 94 in the embodiment of the knee joint endoprosthesis apparatus 12 of FIGS. 1 to 8, namely by the length of the coupling members 98 measured from the coupling projection top side 62.

To form the groove 158, the meniscal component 18 is machined with a milling tool starting from the bottom side 42. In this way, an arcuate perforation 166 corresponding to the groove 158 is prepared in the meniscal component 18.

Commencing from the separating position, which is schematically depicted in FIG. 14, the meniscal component 18 and the tibial component 16 are brought into engagement with one another by displacing same in parallel to the abutment plane 34. The free ends 114 and 116 with the coupling members 98 are inserted through the insertion opening 60 between the delimiting faces 64 and 66.

The insertion width 74 is somewhat smaller than the diameter 88 of the second portion 102, such that the coupling elements 104 and 106 are pivoted somewhat in the direction toward the longitudinal axis 96 upon inserting the coupling projection 54 through the insertion opening 60.

As soon as the first portion 100 engages behind the two lobes 152 and 154 on the meniscal component 18, the knee joint endoprosthesis apparatus 12 adopts the intermediate position.

By moving the meniscal component 18 in the direction toward the top side 32 of the tibial component 16, the coupling members 98 dip into the coupling member receptacle 160 and secure the meniscal component 18 to the tibial component 16. The bottom side 42 of the meniscal component 18 in the coupling position, which is schematically depicted in FIGS. 13, 16, and 17, then abuts against the top side 32 of the tibial component 16.

The rotation limiting device 138 of the knee joint endoprosthesis apparatus 12 is configured as in the embodiment of the knee joint endoprosthesis apparatus 12 of FIGS. 1 to 8.

The coupling members 98 engaging into the coupling member receptacle 160, depending on the selection of the angular range 162, can also contribute to the limiting of a rotation of the tibial component 16 and the meniscal component 18 relative to one another. Due to the groove 158 being configured concentrically to the longitudinal axis 96, the coupling members 98 in cooperation with the groove 158 also guide the rotational movement of the tibial component 16 and the meniscal component 18 relative to one another.

For separating the meniscal component 18 from the tibial component 16, the meniscal component 18, as with the other embodiments described above, must again be slightly raised from the top side 32, such that the coupling members 98 and the coupling member receptacle 160 are out of engagement. From this intermediate position, the meniscal component 18 and the tibial component 16 can then be displaced relative to one another in parallel to the abutment plane 34 and the coupling projection 54 and the coupling receptacle 56 can be brought out of engagement.

The described embodiments of knee joint endoprosthesis apparatuses 12 enable an implantation that is gentle to ligaments. It is also possible to exchange the meniscal component 18 in a simple manner as necessary.

1. A knee joint endoprosthesis apparatus comprising a tibial component for anchoring to a tibia, and comprising a meniscal component, wherein the tibial component and the meniscal component are coupled to one another in a coupling position and are completely separated from one another in a separating position, which tibial component has a top side defining an abutment plane, against which top side a bottom side of the meniscal component abuts in the coupling position, wherein the knee joint endoprosthesis apparatus comprises a coupling device for coupling the tibial component and the meniscal component in the coupling position, wherein the knee joint endoprosthesis apparatus is transferrable from the separating position into the coupling position by a relative movement of the tibial component and the meniscal component, wherein the coupling device is configured in such a way that the tibial component and the meniscal component, upon transferring the knee joint endoprosthesis apparatus from the separating position into the coupling position, are displaceable relative to one another in a direction parallel to the abutment plane from the separating position into an intermediate position and from the intermediate position in a direction transverse to the abutment plane and toward one another into the coupling position.

2. The knee joint endoprosthesis apparatus in accordance with claim 1, wherein the coupling device comprises at least one first coupling element and at least one second coupling element, wherein the at least one first coupling element is arranged or formed on the tibial component or on the meniscal component, wherein the at least one second coupling element is arranged or formed on the meniscal component or on the tibial component, wherein the at least one first coupling element and the at least one second coupling element are out of engagement in the separating position, and wherein the at least one first coupling element and the at least one second coupling element are in engagement in the coupling position.

3. The knee joint endoprosthesis apparatus in accordance with claim 2, wherein the at least one first coupling element is configured in the form of a coupling projection, wherein the at least one second coupling element is configured in the form of a coupling receptacle with a coupling recess, wherein the coupling receptacle is configured to accommodate the coupling projection in the intermediate position, and wherein the coupling recess is configured to accommodate at least a part of the coupling projection in the coupling position.

4. The knee joint endoprosthesis apparatus in accordance with claim 3, wherein the coupling recess is open in a direction perpendicular to the abutment plane.

5. The knee joint endoprosthesis apparatus in accordance with claim 3 , wherein the coupling recess is open exclusively in a direction perpendicular to the abutment plane.

6. The knee joint endoprosthesis apparatus in accordance with claim 1, wherein the meniscal component comprises the coupling recess, which is open facing in the direction toward the tibial component for inserting the at least one part of the coupling projection as the result of a relative movement from the intermediate position into the coupling position in the direction transverse to the abutment plane.

7. The knee joint endoprosthesis apparatus in accordance with claim 1, wherein the meniscal component has an insertion opening for inserting the coupling projection into the coupling receptacle of the meniscal component.

8. The knee joint endoprosthesis apparatus in accordance with claim 7, wherein the coupling projection has a coupling projection top side facing away from the abutment plane, wherein the insertion opening defines an upper delimiting face and a lower delimiting face, which extend in parallel to the bottom side of the meniscal component, wherein a distance of the lower delimiting face from the bottom side of the meniscal component is smaller than a distance of the upper delimiting face from the bottom side of the meniscal component, and wherein the distance of the lower delimiting face from the bottom side of the meniscal component is smaller than a distance of the coupling projection top side from the abutment plane.

9. The knee joint endoprosthesis apparatus in accordance with claim 7, wherein the insertion opening defines an insertion width, wherein the coupling projection defines a coupling projection width, and wherein the coupling projection width corresponds to at least the insertion width.

10. The knee joint endoprosthesis apparatus in accordance with claim 9, wherein the coupling projection width is greater than the insertion width.

11. The knee joint endoprosthesis apparatus in accordance with claim 9, wherein at least first and/or second moveable coupling elements are arranged or formed on the coupling projection and/or on the insertion opening, and wherein the coupling elements upon the transition from the separating position into the intermediate position are moveable for temporarily at least one of: reducing the coupling projection width and enlarging the insertion width.

12. The knee joint endoprosthesis apparatus in accordance with claim 11, wherein the at least one first and/or second coupling elements are arranged or formed so as to be moveable in parallel to the abutment plane.

13. The knee joint endoprosthesis apparatus in accordance with claim 11 , wherein the at least one first and/or second coupling elements are deflectable against the action of a restoring device from a coupling element base position, in which the restoring device does not exert a restoring force on the at least one first and/or second coupling elements, into a coupling element deflected position.

14. The knee joint endoprosthesis apparatus in accordance with claim 13, wherein the restoring device comprises at least one restoring element, and wherein the at least one restoring element is associated with the at least one first and/or second coupling element.

15. The knee joint endoprosthesis apparatus in accordance with claim 14, wherein the at least one first and/or second coupling element comprises the at least one restoring element.

16. The knee joint endoprosthesis apparatus in accordance with claim 14, wherein a respective restoring element is associated with each of the at least one first and/or second coupling elements.

17. The knee joint endoprosthesis apparatus in accordance with claim 1, wherein the at least one restoring element is configured in the form of a spring element.

18. The knee joint endoprosthesis apparatus in accordance with claim 3, wherein the coupling projection comprises at least one coupling member, wherein the coupling receptacle comprises at least one coupling member receptacle, and wherein, in the coupling position, the at least one coupling member engages into the at least one coupling member receptacle.

19. The knee joint endoprosthesis apparatus in accordance with claim 18, wherein the at least one coupling member is arranged or formed projecting from the at least one coupling element.

20. The knee joint endoprosthesis apparatus in accordance with claim 18, wherein the at least one coupling member projects facing away from the at least one coupling element in a direction transverse to the abutment plane.

21. The knee joint endoprosthesis apparatus in accordance with claim 1, wherein the coupling member receptacle is configured in the form of a coupling member receptacle groove that is open facing in the direction toward the abutment plane.

22. The knee joint endoprosthesis apparatus in accordance with claim 3, wherein the coupling projection comprises at least one portion, which extends directly from the top side of the tibial component, and a second portion adjoining the first portion, and wherein a maximum extent of the first portion in a plane parallel to the abutment plane is smaller than a maximum extent of the second portion in a plane parallel to the abutment plane.

23. The knee joint endoprosthesis apparatus in accordance with claim 1, wherein the meniscal component and the tibial component in the coupling position are moveable relative to one another.

24. The knee joint endoprosthesis apparatus in accordance with claim 1, wherein the coupling device forms a rotary bearing device for rotatably bearing the meniscal component and the tibial component relative to one another in the coupling position.

25. The knee joint endoprosthesis apparatus in accordance with claim 24, wherein the rotary bearing device comprises the at least one first coupling element and the at least one second coupling element.

26. The knee joint endoprosthesis apparatus in accordance with claim 2, wherein the at least one first coupling element and the at least one second coupling element are formed rotationally symmetrical or substantially rotationally symmetrical and define an axis of rotation, in relation to which the meniscal component and the tibial component in the coupling position are rotatably mounted relative to one another.

27. The knee joint endoprosthesis apparatus in accordance with claim 26, wherein the axis of rotation extends transversely to the abutment plane.

28. The knee joint endoprosthesis apparatus in accordance with claim 26, wherein the at least one first and/or second coupling elements are arranged or formed so as to be at least one of displaceable relative to the axis of rotation and pivotable relative the axis of rotation.

29. The knee joint endoprosthesis apparatus in accordance with claim 26, wherein the coupling member receptacle groove is arranged or formed concentrically surrounding the axis of rotation.

30. The knee joint endoprosthesis apparatus in accordance with claim 22, wherein an undercut that is open in parallel to the abutment plane is formed between the top side of the tibial component and the second portion in the region of the first portion and faces away from the axis of rotation.

31. The knee joint endoprosthesis apparatus in accordance with claim 3, wherein the coupling receptacle has at least one undercut that is open in parallel to the bottom side of the meniscal component and faces in the direction toward the axis of rotation, said undercut facing toward the axis of rotation.

32. The knee joint endoprosthesis apparatus in accordance with claim 1, wherein the knee joint endoprosthesis apparatus comprises a rotation limiting device for limiting a rotation of the meniscal component and the tibial component relative to one another in the coupling position.

33. The knee joint endoprosthesis apparatus in accordance with claim 32, wherein the rotation limiting device comprises at least one first stop face arranged or formed on the tibial component and at least one second stop face that cooperates therewith and is arranged or formed on the meniscal component, and wherein the at least one first stop face and the at least one second stop face abut against one another in a rotation position deflected from a base position by a maximum rotation angle.

34. The knee joint endoprosthesis apparatus in accordance with claim 33, wherein the at least one first stop face and the at least one second stop face extend transversely to the abutment plane.

35. The knee joint endoprosthesis apparatus in accordance with claim 1, wherein the meniscal component has a meniscal component top side, which has medial and lateral meniscal joint faces.

36. A knee joint endoprosthesis comprising a knee joint endoprosthesis apparatus and a femoral component that cooperates therewith, wherein the knee joint endoprosthesis apparatus is configured in the form of a knee joint endoprosthesis apparatus in accordance with claim 1.

37. The knee joint endoprosthesis in accordance with claim 36, wherein the femoral component comprises medial and lateral femoral joint faces facing in the direction toward the meniscal component, which femoral joint faces in an implantation position of the knee joint endoprosthesis abut against medial and lateral meniscal joint faces of the meniscal component and cooperate therewith upon a relative movement of the femoral component and the meniscal component.
